(19) United States
(12) Patent Application Publication
(10) Pub. No.: US 2010/0239466 A1
(43) Pub. Date: Sep. 23, 2010

(54) Surface Plasma Gas Processing

(75) Inventors: Antoine Rousseau, Paris (FR); Katia Allegraud, Enghien Les Bains (FR); Olivier Guaitella, Bures Sur Yvette (FR)

Correspondence Address: MILLER, MATTHIAS & HULL, ONE NORTH FRANKLIN STREET, SUITE 2350, CHICAGO, IL, 60606, US

(73) Assignees: Ecole Polytechnique, Paris (FR); Centre National De La Recherche Scientifique -CRNS-, Paris (FR)

(21) Appl. No.: 12/652,857

(22) Filed: Jan. 6, 2010

(30) Foreign Application Priority Data
Jul. 6, 2007 (FR) 0704900

(51) Int. Cl. A61L 9/22; B32B 37/02; B32B 38/04; B32B 38/14; B01J 19/08; A61L 9/16; B01J 37/34; B01J 35/00

(52) U.S. Cl. 422/121; 156/60; 156/250; 156/277; 422/186.04; 422/180

(57) ABSTRACT

The invention relates to a gas processing unit adapted for generating a surface plasma in the vicinity of a photocatalyst, that has a planar configuration. The photocatalyst is deposited in the form of a thin layer on a dielectric substrate and at least one plasma supply electrode is formed above the photocatalyst thin layer. Such a configuration increases the interaction between the plasma and the photocatalyst. The unit can be used for a gas processing of the pollution-control, odour reduction or bactericidal treatment type with a high efficiency.

### CROSS-REFERENCE TO RELATED APPLICATION

This application is a U.S. national stage filing of International patent Application No. PCT/FR2008/051137 Filed on Jun. 24, 2008, which claims priority under the Paris Convention to French Application No. 07 04900, filed on Jul. 6, 2007.

### FIELD OF THE DISCLOSURE

The present invention relates to a surface plasma gas processing unit, a device that comprises at least one such unit and a method of manufacturing the processing unit.

### BACKGROUND OF THE DISCLOSURE

Anti-pollution standards are being implemented, in particular at the European level, which are ever more restrictive. These standards are imposed on manufacturers, who must incorporate them into the design of new production units. They are also a response to the population's growing preoccupation with environmental issues, while the standard of living is improving steadily. However, the pollution-control methods that are being used or intended to be used must consume as little energy as possible, in particular when the concentration of pollutants in the effluents to be processed is low or very low. In such cases, the pollution-control methods must be all the more efficient, the more diluted the pollutants are.

The pollution-control methods that are already being used can be classed into two categories, depending on whether they use a plasma or not.

The methods that use thermal cracking of the pollutants, a catalytic reaction, adsorption or cryo-condensation of the pollutants, or also those that use biofilters, do not use plasma. They mainly use techniques for oxidizing the pollutants, or techniques for trapping them. The oxidation techniques, whether they are purely thermal (thermal cracking) and/or performed in the presence of a catalyst (catalytic oxidation), require the gas to be heated to several hundred degrees. They are effective mainly for high concentrations of pollutants. But their energy costs are very high, and become prohibitive when the pollutants to be treated are present in low concentrations. It can be necessary to remove even very diluted pollutants, in particular when these are particularly toxic or harmful to the environment. The trapping (or adsorption) techniques generally require expensive maintenance. Moreover, they do not remove the pollutants, but merely allow for them to be extracted by fixing them on a support. When the support is saturated, it must be changed. The support is then difficult to handle, because of the large quantity of pollutants that it then contains. The problem then arises of the reprocessing of the support or its storage in landfill or destruction when it cannot be recycled. Finally, the biofilters contain microorganisms that are capable of consuming volatile organic compounds. The pollutants in question are then effectively removed, but the biofilters are themselves very bulky and require complex maintenance in order to keep the microorganisms alive.

In comparison, the methods that use a plasma have lower energy and maintenance costs. In particular, the use of plasmas at atmospheric pressure is compatible with a large number of applications. In particular, so-called cold plasmas make it possible to destroy several pollutant species inexpensively. But they often lead to the production of toxic compounds such as, in particular, nitrogen oxides (NOₓ), carbon monoxide (CO) or other volatile organic compounds.

In order to overcome these drawbacks, it has been proposed that the use of a plasma be combined with the use of a catalyst, which makes it possible to better control the chemical reactions being used, and therefore to select cracking products that are not themselves toxic or pollutant. The devices known as “packed bed reactors” were developed first. They are comprised of a cylindrical reactor that is filled with beads of a catalyst material. But they are bulky, and produce an important pressure loss when a gas stream to be processed is circulated in the reactor. Moreover, their configuration makes them unsuitable for arrangements in series or in parallel, in particular in order to process larger gas streams.

In addition, dielectric-barrier discharge devices have been developed. In such devices, the occurrence of an electric arc is inhibited by means of a dielectric insulator placed between the discharge electrodes. It is not then necessary for the geometry of the electrodes to be asymmetric in order to maintain a constant supply of cold plasma, unlike ring discharges. However, the power supply cannot be direct (DC) since electrical charges accumulate very rapidly on the dielectric and destroy a stationary electric field that is generated between the electrodes. A plasma that is thus powered by a direct voltage in the presence of a dielectric barrier is therefore extinguished immediately. But the advantages of these dielectric-barrier discharge devices are numerous:
the dielectric barrier provides greater security and a more homogeneous distribution of the plasma;
a cooling fluid circuit can optionally be incorporated into the dielectric barrier;
this type of reactor can easily be expanded, in particular in order to obtain processing capacities that are suited to industrial applications; and
they are compatible with operation at atmospheric pressure.

For these reasons, dielectric-barrier discharge devices are widely used. These uses include the production of ozone, surface treatments, the production of ultraviolet radiation in excimer lamps and the production of infrared radiation in CO₂ lasers. A great number of geometries have been developed for these devices, depending on the specific features of each use. In particular, the surface configuration known as “One Atmosphere Uniform Glow Discharge Plasma” (OAUGDP) was developed to generate a flow of gas. The configuration known as “creeping discharge” was developed initially for laser applications, but it is also used to produce stable plasmas that extend over large areas, without electric arcs appearing.

Finally, several devices have also been developed more recently that combine the use of a dielectric-barrier discharge and the use of a catalyst, in particular a photocatalyst. An example of this type of device, which has been designed in particular for cracking toluene, comprises an electrode wire that passes through a cylinder filled with glass beads, themselves covered with titanium oxide (TiO₂) in anatase form.

Document US 2005/0118079 describes another device of this type, which is adapted for producing a surface discharge and which has a planar configuration for the electrodes. Beads of a photocatalyst material are placed above one of the electrodes, which is exposed to the gas to be processed. However, in this configuration of the electrodes and photocatalyst material, the plasma produced by the surface discharge is only partially in contact with the photocatalyst material. For this reason, the device only has limited efficiency for processing gas streams.

### SUMMARY OF THE DISCLOSURE

An object of the present invention is to propose a gas processing device, for example for eliminating pollutants present in this gas, which does not have the drawbacks of the earlier systems mentioned above.

More particularly, the object of the invention is to propose a gas processing device with low manufacturing costs, the energy consumption of which is low during operation, which has a high processing efficiency, and which is compatible with large volumes of gas to be processed.

To this end, the invention proposes a gas processing unit, which comprises:
a dielectric support that has an active face and a rear face which are parallel;
a first electrode which is carried by the active face of the support;
a second electrode which is carried by the rear face of the support, and which is offset with respect to the first electrode along a direction parallel to the support; and
at least one portion of a photocatalyst which is arranged above the active face of the support, and which is able to activate the processing of the gas when said photocatalyst receives radiation.

The unit is adapted for forming a surface plasma above the active face of the support, in a zone extending from the first electrode towards the second electrode when these electrodes are connected to two terminals of a power source. The plasma then produces the radiation which is received by the photocatalyst.

According to the invention, the photocatalyst portion is a thin layer which is located on the active face of the support, and the first electrode is arranged over at least part of the thin photocatalyst layer, on a side of the latter that is opposite the support.

Within the context of the invention, by thin layer is meant a configuration of a portion of material in which the portion has a thickness that is much smaller than the other dimensions of this portion. The portion is produced on a support using a deposition process where the material is brought onto a surface of the support in gas, liquid or even plasma form. In particular, such a thin layer can be formed from molecules, atoms, clusters of atoms, a liquid film, droplets, etc., which are brought onto the substrate individually. The use of such thin layer deposition process for the photocatalyst material of a processing unit according to the invention is particularly cost-effective, and makes it possible to manufacture the unit at a low cost price.

In addition, a processing unit according to the invention may have any dimensions, which can be selected depending on the flow rate of the gas to be processed. In particular, large units, which are suitable for industrial uses, can be produced simply. The different components that constitute the processing unit can be manufactured easily in large sizes, including the thin-layer photocatalyst portion.

Gas processing carried out using a unit according to the invention combines the use of a cold surface plasma at atmospheric pressure and the use of a photocatalyst. The surface plasma constitutes a stable source of the radiation that activates the catalyst. The catalyst then in turn activates a conversion of pollutants that are brought into contact with it. The gas processing carried out in this way may be an at least partial pollution-control of the gas, in particular with respect to volatile organic compounds that are initially present in this gas, an odour reduction of the gas, a bactericidal treatment, or a combination of at least two of these treatments. In addition, given that the processing unit is light and small, it can easily be installed in a workshop, home, vehicle, aeroplane or submarine, in particular by being integrated into a ventilation or air-conditioning system. Finally, it is adapted for carrying out gas processing in both open and closed or confined environments.

The processing unit of the invention has a particularly high processing efficiency. As a result of the configuration of the photocatalyst as a thin layer supported by the substrate, which also supports the electrodes, the photocatalyst has a large contact area with the electric discharge plasma. At the same time, the photocatalyst has a very high surface-to-volume ratio. In this way, the interaction of the plasma with the photocatalyst is highly developed. This interaction is promoted even more by the positioning of the first electrode over the photocatalyst layer. To this end, one edge of the first electrode, which is oriented towards the plasma zone, may be arranged over the thin photocatalyst layer. Thus, the surface plasma is generated directly at the surface of the photocatalyst layer, from the edge of the electrode.

In addition, a processing unit of the invention can be produced particularly simply when the thin photocatalyst layer is continuous between the first electrode and the support. In this case, the first electrode is formed on the thin photocatalyst layer, without the prior removal of parts of the photocatalyst.

The photocatalyst used in this way allows in particular for an efficient cracking of the volatile organic compounds, without producing waste molecules that are themselves toxic or polluting, or producing them in proportions that are small enough not to have a harmful effect.

Another particularly noteworthy advantage of the invention is that, given that the thin photocatalyst layer is arranged at least partly between the first electrode and the substrate, it causes a reduction in the starting voltage of the surface discharge plasma. The photocatalyst layer has a dielectric permittivity that is generally higher than that of the support, so that it contributes to the strengthening of the active electric field that is effective upon the start of the plasma. Given that the start voltage of a plasma is generally greater than the electrical voltage that is subsequently necessary to maintain this plasma, this reduction in the start voltage makes it possible to considerably simplify the electric power source which is used with the processing unit. The overall cost of installation and use of a gas processing device that incorporates a unit according to the invention is reduced as a result. In addition, the electromagnetic noise generated by the device is also reduced.

Several improvements to the invention may be introduced in various embodiments, separately or by combining some of them. These improvements include the following two:
the processing unit may be further adapted for forming another surface plasma at the rear face of the support, at least one other portion of a photocatalyst being arranged on this rear face and able to activate the processing of a gas when this other photocatalyst portion receives radiation produced by the other plasma; and
a third electrode may be carried by the active face of the support, which is offset with respect to the second electrode in the direction parallel to the support along an opposite direction to the first electrode, and which is capable of increasing the plasma zone when an electrical voltage is also applied between the second and third electrodes.

The invention moreover proposes a gas processing device, which comprises:
at least one processing unit, as described previously;
gas flow conduct means, which are capable of conducting the gas onto the thin photocatalyst layer in the plasma zone; and
an electric power source, which is connected to the first and second electrodes.

Such device, which is intended to operate with a gas to be processed, is particularly light and easy to install. In particular, given that the gas is processed directly at atmospheric pressure, no pump or airtight low-pressure pipe is needed.

Preferably, the electric power source is adapted for producing a signal that may vary cyclically between two opposing polarities. In this way, accumulations of electrostatic charges that are likely to appear on some parts of the processing unit, in particular on the dielectric support and/or on the photocatalyst portion, are reduced or neutralized. Continuous operation of the processing unit is then facilitated.

If necessary, depending on the dimensions of each processing unit in relation to the flow rate of the gas to be processed, several substantially identical processing units may be positioned side-by-side in parallel in the device, so that two neighbouring units are separated by a distance that is adapted for these two units together form part of the gas flow conduct means.

Finally, the invention proposes a method of manufacturing a gas processing unit, which comprises the following steps:
/1/ providing a support film with two parallel faces;
/2/ depositing a layer of photocatalyst on at least one of the faces of the support film, by using a tool for depositing a thin layer of this photocatalyst; and
/3/ positioning at least a first and a second electrically conducting portion on the two faces of the support film, respectively, these conducting portions being offset along a direction parallel to the film, and one of the two portions being arranged at least partly over the thin photocatalyst layer.

Such a method may be used to manufacture a gas processing unit such as described previously. The conducting portions that are arranged in step /3/ form the first and second electrodes introduced above.

In order to obtain a particularly low cost price for the processing unit, the support film may initially have a length that corresponds to several processing units. Step /2/ is then carried out continuously while the support film is being translated in the tool for depositing the thin photocatalyst layer. Then the support film is cut to dimensions that correspond individually to separate processing units. Such continuous method has a particularly high output and manufacturing rate, in particular because it obviates the need to readjust the thin-layer deposition tool each time a portion of this film that is intended for a new gas processing unit is produced.

### BRIEF DESCRIPTION OF THE DRAWINGS

Other features and advantages of the present invention will become apparent in the following description of non-limitative embodiments, with reference to the attached drawings, in which:

FIGS. 1a and 1b are cross-sectional and plan views respectively of a gas processing unit according to a first embodiment of the invention;

FIGS. 2a and 2b correspond to FIGS. 1a and 1b respectively for a second embodiment of the invention;

FIG. 3 is a perspective schematic diagram of a gas processing device that incorporates several processing units according to the invention.

### DETAILED DESCRIPTION OF THE DISCLOSURE

It is understood that, for clarity sake, the components shown in these figures are not in proportion to actual dimensions or ratios of actual dimensions. For these dimensions, reference will be made to the indications given below. In addition, identical reference numbers indicated on different figures refer to the identical components or components that have identical functions.

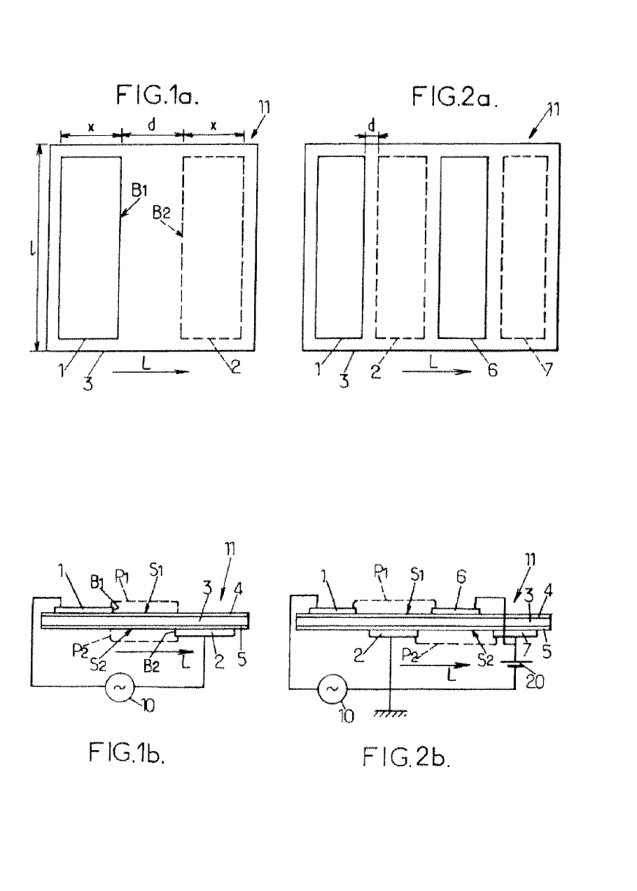

According to FIGS. 1a and 1b, a gas processing unit, which is denoted 11 as a whole, comprises a support 3 made from a dielectric material. This support 3 is preferably planar, for example square with side dimensions that can be 60 cm×60 cm for example. It has a reduced thickness that may be a few tenths of millimetres to a few millimetres, in particular. Optionally, the support 3 may have a layered structure. It may comprise a glass sheet that has a thickness comprised between 0.4 and 2.0 mm (millimetres). Alternatively, it may comprise a film made from an organic material, which may be based on polycarbonate, polyethylene terephthalate or polyimide for example. In this case, the organic film has preferably been passivated to prevent it from being degraded while the unit 11 is in operation.

The two faces of the support 3 are marked S1 and S2. At least one of them is covered with a thin layer of a photocatalyst. In the embodiment of the invention described here, each of the faces S1 and S2 is covered with a thin layer of titanium oxide (TiO₂) denoted 4 or 5 respectively. Other photocatalyst materials may be used for the layers 4 and 5, such as zinc oxide (ZnO), nickel oxide (NiO), chromium oxide (Cr₂O₃), zirconia (ZrO₂), cerium oxide (CeO₂), etc. Such photocatalyst materials have a redox efficiency that is particularly suited to carrying out the cracking of pollutant compounds by oxidizing them. The layers 4 and 5 may each have a thickness comprised between 10 nm (nanometres) and 100 μm (micrometres), preferably between 50 nm and 200 nm.

Optionally, the support 3 may itself comprise a substrate and a base layer that it carries on one of the two faces S1 and S2, or on both of these faces. Such a base layer, which is not shown in the figures, forms at least the active face S1 of the support 3 and carries the layer 4. Such a base layer may improve the adhesion of the layer 4 to the substrate, and/or adapt the texture of the layer 4 of photocatalyst material, for example its roughness or porosity, in order to increase the processing efficiency of the unit 11.

The layers 4 and 5, as well as the optional base layer, are deposited on the substrate or the support 3 using an appropriate thin-layer deposition tool, such as a cathode sputtering deposition unit for example. Such deposition tool and its operation are known to a person skilled in the art and are not described again in detail here. When the support 3 is in the form of a flexible film, the tool for depositing the thin layer(s) of photocatalyst 4 (and 5) may be arranged downstream of a supply roll of the support film 3, which is unwound. Such a deposition unit is commonly called a “roll coater” and makes high production rates possible.

Two parallel conducting portions 1 and 2 are formed on the faces S1 and S2 respectively. For example, the portions 1 and 2 are made of conducting tin oxide (SnOₓ), silver (Ag) or any other stable conducting material, and may have thicknesses comprised between a few nanometres and several tens of micrometres. They extend substantially over the whole width I of the support 3 and their dimension x in a common direction L, which is parallel to the faces S1 and S2 and perpendicular to the width I of the support 3, may be 10 cm. At least one of these conducting portions 1 and 2 may be formed using a tool for depositing a thin layer of an electrically conducting material. Alternatively, it may be formed by screen printing.

The portions 1 and 2 are offset with respect to each other along the direction L, so that each of the portions 1, 2 has an edge which is oriented towards the other portion. These edges are called internal edges of the portions 1 and 2 and are denoted B1 and B2 respectively. They are separated by a distance d that may be comprised between a few millimetres and ten centimetres, parallel to the faces S1 and S2.

Under these conditions, when the conducting portions 1 and 2 are connected to two respective terminals of an AC electric power source 10 (FIG. 1b), a discharge appears on each face S1, S2. This discharge generates a surface plasma that extends from the internal edge B1, B2 of each conducting portion 1, 2 towards the other conducting portion. In FIG. 1b, the reference numbers P1 and P2 denote the volumes of these surface plasmas. The portions 1 and 2 then act as supply electrodes for each plasma. For this reason, they are hereafter called electrodes 1, 2. The power supply 10 may be adapted to provide a low output voltage, typically 3 kV (kilovolt) and possibly less than 1 kV for certain particular geometries. This voltage may be sinusoidal or pulsed, with a frequency comprised between a few hertz and a few hundreds of kilohertz.

Each electrode 1, 2 is formed over the corresponding layer of photocatalyst material 4, 5. Preferably, at least the internal edge B1, B2 of each portion 1, 2 is situated over the corresponding layer 4, 5. The parts of the layers 4 and 5 that are comprised between the electrodes 1 and 2, in projection, then have their uncovered surfaces which are situated within the volumes of plasma P1 and P2. For this reason, the photocatalytic activity of the layers 4 and 5 is used to the maximum.

Preferably, at least part of the thin photocatalyst layer 4 which is located between the support 3 and the electrode 1 has a relative dielectric permittivity greater than 6.0. The result of this relative dielectric permittivity value being greater than that of the support 3 is a reduction in the apparent electrical thickness that is present between the electrodes 1 and 2 when they are powered electrically. The minimum voltage necessary to start initially the surface plasmas is then reduced. In the present embodiment of the invention, the two layers 4 and 5 are identical, and therefore contribute in an identical manner to the reduction in the start voltage of the surface plasmas.

Optionally, a third conducting portion 6 may be arranged on the face S1, as shown in FIGS. 2a and 2b. This portion 6 may also be identical to the electrodes 1 and 2, and offset with respect to the electrode 2 along the direction L in the opposite direction to the electrode 1. The electrode 6 is intended to be polarized electrically with respect to the electrodes 1 and 2 using a direct voltage source 20 of a few hundred volts. This makes it possible to increase the extension of the surface plasma formed on the face S1, parallel to the direction L, compared to the extension that would result only from the separating distance d between the electrodes 1 and 2. In this way, a larger area of the layer of photocatalyst material 4 is situated in the plasma volume P1, and is therefore activated by the radiation produced by this plasma. The electrical discharge obtained is then called creeping discharge, in contrast to a unit 11 with only two electrodes, as shown in FIGS. 1a and 1b.

The fourth electrode 7 has, vis-à-vis the electrode 2 and the surface plasma generated on the face S2, a function identical to that of the electrode 6 vis-à-vis the electrode 1 and the face S1.

A unit 11 that corresponds to FIGS. 2a and 2b may alternatively be used as a union of two units that are each similar to the unit in FIGS. 1a and 1b, and which are juxtaposed in the direction L. In this case, the electrode 6 has a function that is identical to that of the electrode 1, and is connected with this latter to the first terminal of the AC power source 10. Similarly, the electrode 7 has a function identical to that of the electrode 2, and it is connected with it to the second terminal of the source 10. The polarization voltage source 20 is eliminated.

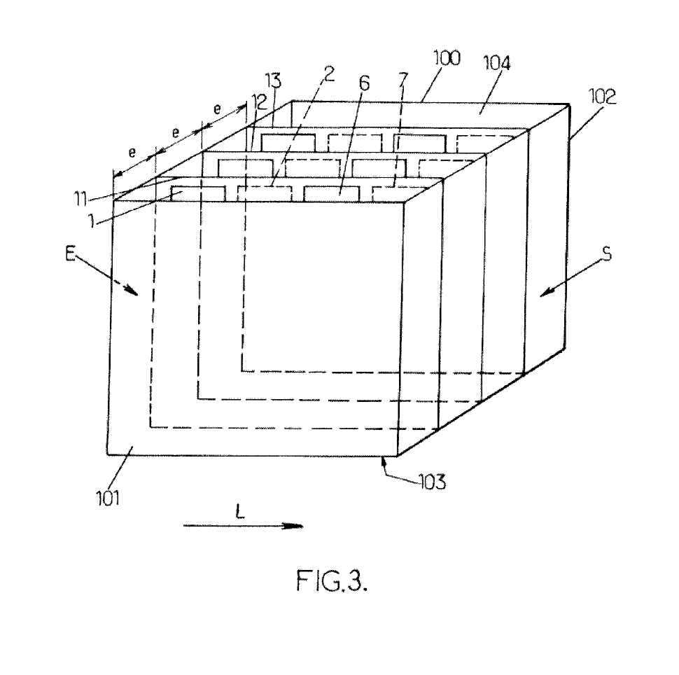

Such unit 11 may be used to reduce the concentration of pollutants that are present in a surrounding atmosphere. These pollutants may be volatile organic compounds in particular. To this end, several units 11-13 may be positioned parallel to each other in a frame 100 of a gas processing device (FIG. 3). This frame 100, which may have a parallelepipedal shape, is open on two faces E and S which are opposite each other, for example in the direction L. The other faces 101-104 of the frame 100 are closed with panels. A gas stream can then enter the device by the face E and exit by the face S. Between the two faces E and S, the gas stream flows between the units 11-13 which help to conduct it, being separated by a gap e that may be comprised between a few millimetres and a few centimetres. Optionally, several units may be positioned in line with each other, so that the gas stream is processed successively by these units during the same flow through the device.

The electrodes 1 and 6, on the one hand, and 2 and 7, on the other hand, of each unit 11-13 are connected respectively to the two terminals of the AC power supply 10 in FIG. 1a (not shown in FIG. 3). Surface plasmas are then produced simultaneously on large portions of the two faces S1 and S2 of all of the units 11-13.

Such a device is adapted performing the processing of the surrounding air, operating at atmospheric pressure. Optionally, a fan (not shown) may be positioned at the level of one of the faces E and S, in order to bring about the flow of the gas stream through the frame 100, between the units 11-13. As a result of the configuration of each unit 11-13, significant contact is obtained between the gas that is intended to be processed and the surface plasmas that are generated. In particular and in the manner that has already been explained above, the configuration of the internal edges of each electrode, over the underlying layer of photocatalyst material, promotes a synergy between the surface plasma and the photocatalyst. On each face of the units 11-13, the plasma extends over the whole length of the electrodes. Thus, the device has a high pollution-control efficiency. In addition, its energy consumption, which is determined by the electrical power provided by the supply 10, is low.

Finally, the use of the photocatalyst material of the layers 4 and 5 makes it possible to reduce the formation of undesirable cracking products, by promoting a selected chemical method. In particular, the use of titanium oxide makes it possible to increase the proportion of carbon dioxide (CO₂) in the processed gas, for a large number of polluting organic compounds initially present. Such cracking of the pollutants corresponds to a complete oxidation thereof.

It is understood that the embodiments just described in detail may be adapted in many ways, in particular depending on the application being considered, while retaining at least some of the advantages of the invention. These adaptations include:
the electrodes 1 and 2 may be very thin, with a width x of the order of one millimetre, and offset with respect to each other by a distance d of a few millimetres. Each support 3 may then comprise a large number of pairs of electrodes 1, 2 that are arranged successively along the flow of the gas stream;
the use of an appropriate catalyst makes it possible to increase locally the concentration of the pollutants at the surface of the support. Even more efficient processing of the gas is obtained, in particular when the pollutants are present in very low concentrations;
the processing of the gas may be carried out continuously or sequentially, depending on whether the gas is admitted in the form of a continuous stream or whether fixed volumes of gas are processed successively, each being enclosed in the processing device for a given duration;
the gas processing device may also comprise a section for the cracking of ozone molecules that may be produced while the gas is being processed by the surface plasmas. Such a section may incorporate a porous material such as manganese oxide (MnO₂) or alumina (γ-Al₂O₃) for example; and
the flow of the gas to be processed may be oriented in any manner parallel to the support 3 of each unit, in relation to the offset direction L of the electrodes 1 and 2.

1. A gas processing unit comprising:
a dielectric support having an active face and a rear face parallel to said active face;
a first electrode carried by the active face of the support;
a second electrode carried by the rear face of the support, and offset with respect to the first electrode along a direction parallel to the support; and
at least one portion of a photocatalyst arranged above the active face of the support and able to activate the processing of the gas when said photocatalyst receives radiation,
said unit being adapted for forming a surface plasma above the active face of the support, in a zone extending from the first electrode towards the second electrode when said first and second electrodes are connected to two terminals of an electric power source, and said plasma producing radiation that is received by the photocatalyst;
the unit being characterized in that the photocatalyst portion is a thin layer located on the active face of the support,
and in that the first electrode is arranged over at least part of the thin photocatalyst layer, on a side of said thin layer opposite the support.

2. The unit according to claim 1, wherein one edge of the first electrode oriented towards the plasma zone is arranged over the thin photocatalyst layer.

3. The unit according to claim 1, wherein the thin photocatalyst layer is continuous between the first electrode and the support.

4. The unit according to claim 1, wherein the thin photocatalyst layer has a thickness comprised between 10 nanometres and 100 micrometres, preferably between 50 nanometres and 200 nanometres.

5. The unit according to claim 1, adapted so that the processing of the gas is selected from an at least partial pollution-control of said gas, an odour reduction of said gas, a bactericidal treatment, and a combination of at least two of said treatments.

6. The unit according to claim 1, adapted for further forming another surface plasma at the rear face of the support, at least one other portion of a photocatalyst being arranged on said rear face of the support and able to activate a processing of a gas when said other photocatalyst portion receives radiation produced by said other plasma.

7. The unit according to claim 1, further comprising a third electrode carried by the active face of the support, offset with respect to the second electrode along the direction parallel to the support in an opposite direction to the first electrode, and adapted for increasing the plasma zone when an electrical voltage is also applied between the second and third electrodes.

8. The unit according to claim 1, wherein the support has a layered structure.

9. The unit according to claim 1, wherein the support itself comprises a substrate and a base layer carried by said substrate, said base layer forming the active face of the support and carrying the thin photocatalyst layer.

10. The unit according to claim 1, wherein the support comprises a glass sheet having a thickness comprised between 0.4 and 2.0 millimetres, or a film made from an organic material.

11. The unit according to claim 1, wherein the photocatalyst is a material that has a redox efficiency.

12. The unit according to claim 1, wherein part of the thin photocatalyst layer located between the support and the first electrode has a relative dielectric permittivity greater than 6.0.

13. A gas processing device comprising:
at least one processing unit according claim 1;
gas flow conduct means adapted for conducting the gas onto the thin photocatalyst layer in the plasma zone; and
an electric power source connected to the first and second electrodes.

14. The device according to claim 13, comprising several processing units arranged side-by-side in parallel, and two neighbouring units of said device being separated by a distance adapted so that said two units together form part of the gas flow conduct means.

15. A method of manufacturing a gas processing unit, comprising the following steps:
/1/ providing a support film with two parallel faces;
/2/ depositing a layer of a photocatalyst on at least one of the faces of the support film by using a tool for depositing a thin layer of said photocatalyst; and
/3/ arranging at least a first and a second electrically conducting portion on the two faces of the support film, respectively, said conducting portions being offset along a direction parallel to the film, and one of the two portions being arranged at least partly over the thin photocatalyst layer.

16. The method according to claim 15, used to manufacture a gas processing unit according to claim 1.

17. The method according to claim 15, according to which:
the support film has a length corresponding to several processing units;
step /2/ is carried out continuously by translating the support film in the tool for depositing the thin photocatalyst layer; and
the support film is then cut to dimensions corresponding individually to separate processing units.

18. The method according to claim 17, wherein the support film is flexible, and the tool for depositing the thin layer of the photocatalyst used in step /2/ is arranged downstream of a supply roll of said support film, which is unwound.

19. The method according to claim 15, wherein at least one of the conducting portions is formed by using a tool for depositing a thin layer of an electrically conducting material, or by screen printing.
